(19) United States
(12) Patent Application Publication
(10) Pub. No.: US 2015/0018565 A1
(43) Pub. Date: Jan. 15, 2015

(54) METHOD OF PREPARING EZETIMIBE

(71) Applicant: Heading (Nanjing) Pharmaceutical Technologies Co., Ltd., Nanjing (CN)

(72) Inventors: Wensen Li, Holmdel, NJ (US); Laiyue Liu, Yangzhou (CN); Aichen Tang, Yangzhou (CN); Yanmin Wang, Chifeng (CN); Hailong Wang, Nanjing (CN); Wansong Yu, Yangzhou (CN)

(73) Assignee: Heading (Nanjing) Pharmaceutical Technologies Co., Ltd., Nanjing (CN)

(21) Appl. No.: 14/029,327

(22) Filed: Sep. 17, 2013

(30) Foreign Application Priority Data
Jul. 15, 2013 (CN) 201310296900.0

(51) Int. Cl. C07D 205/08

(52) U.S. Cl. CPC C07D 205/08
USPC 548/952

(57) ABSTRACT

A method of preparing ezetimibe. The method includes converting a compound of formula (II) to a compound of formula (III) as shown below:

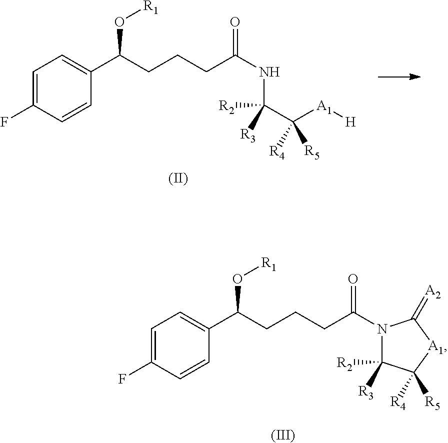

in which R₁-R₅, A₁, and A₂ are defined in the specification.

### CROSS REFERENCE TO RELATED APPLICATION

This application claims priority to Chinese Patent Application No. 201310296900.0, filed Jul. 15, 2013, the content of which is incorporated herein by reference in its entirety.

### BACKGROUND

Ezetimibe, a drug that lowers plasma cholesterol levels, has a chemical structure including as many as three chiral centers. As a result, its synthesis is challenging.

An inefficient process of preparing ezetimibe is described in Thiruvengadam et al., U.S. Pat. No. 5,561,227. It involves a low-yielding stereoselective reduction of a carbonyl group in an azetidinone intermediate to a hydroxyl group in the last step.

A higher yield of ezetimibe can be achieved by another process disclosed in Thiruvengadam et al., U.S. Pat. No. 6,207,822. In this more efficient process, the carbonyl group is stereoselectively reduced to an alcohol product before the formation of an azetidinone intermediate but after the formation of an oxazolidinone intermediate. However, this process uses an expensive stereoselective Corey-Bakshi-Shibata (“CBS”) reducing agent. Further, it is difficult to purify the alcohol product by conventional methods, e.g., crystallization. See People et al., International Application Publication 2005/066120. Its purification by expensive methods, e.g., chiral chromatography, not only increases the costs but also decreases the yield.

There is a need to develop a high-yielding and cost-effective method of stereoselectively preparing ezetimibe.

### SUMMARY

The method of this invention is based on an unexpected discovery of a novel alcohol intermediate useful in preparing ezetimibe, the structure of which is shown below:

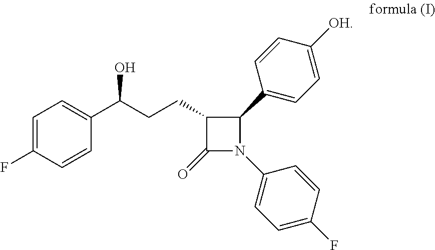 [SMILES: O=C1[C@H](CC[C@H](O)c2ccc(F)cc2)[C@@H](c2ccc(O)cc2)N1c1ccc(F)cc1]

The alcohol intermediate can be purified by an inexpensive method, e.g., crystallization, and can be prepared using an inexpensive stereoselective reducing agent before the formation of an oxazolidinone intermediate.

The method of this invention includes the following steps.

(a) A compound of formula (II) is converted to a compound of formula (III) as shown below:

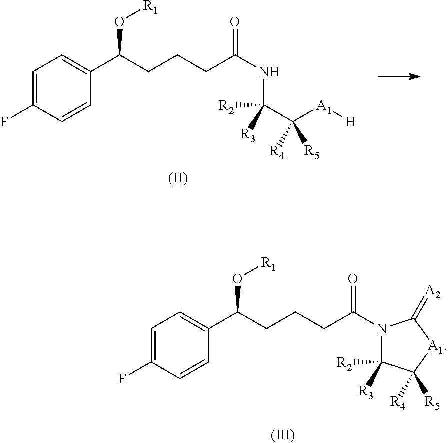

In the above formula, R₁ is H or a protecting group; R₂ is C₁-C₁₀ alkyl, C₂-C₁₀ alkenyl, C₂-C₁₀ alkynyl, C₃-C₁₀ cycloalkyl, C₃-C₁₀ cycloalkenyl, C₁-C₁₀ heterocycloalkyl, C₁-C₁₀ heterocycloalkenyl, aryl, or heteroaryl (preferably, methyl, ethyl, propyl, isopropyl, butyl, isobutyl, tert-butyl, phenyl, benzyl, or diphenylmethyl); each of R₃, R₄, and R₅, independently, is H, C₁-C₁₀ alkyl, C₂-C₁₀ alkenyl, C₂-C₁₀ alkynyl, C₃-C₁₀ cycloalkyl, C₁-C₁₀ heterocycloalkyl, aryl, or heteroaryl (e.g., H), R₂, R₃, R₄, and R₅, together, determining the stereochemistry of step (b) below; A₁ is O, S, or NRₐ, Rₐ being C₁-C₁₀ alkyl, C₂-C₁₀ alkenyl, C₂-C₁₀ alkynyl, C₃-C₁₀ cycloalkyl, C₃-C₁₀ cycloalkenyl, C₁-C₁₀ heterocycloalkyl, C₁-C₁₀ heterocycloalkenyl, aryl, or heteroaryl (preferably, 0 or S); and A₂ is O or S (preferably, O).

(b) A compound of formula (III) is reacted with a compound of formula (IV) to obtain a compound of formula (V) as shown below:

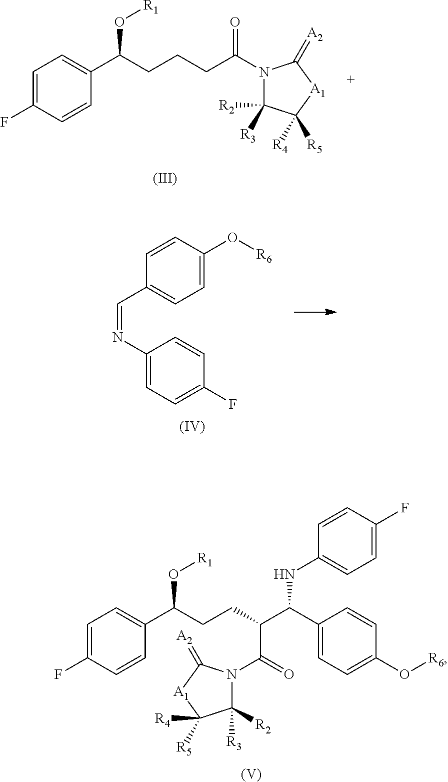

in which R₆ is H or a protecting group.

(c) A compound of formula (V) is cyclized to obtain a compound of formula (VI):

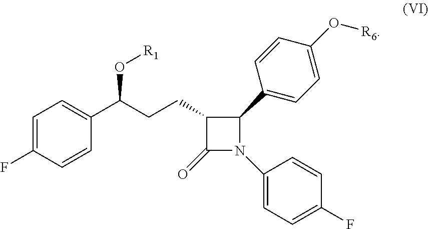

If each of R₁ and R₆ is H, formula (VI) is identical to formula (I). Namely, ezetimibe is obtained in this step.

(d) On the other hand, if either R₁ or R₆ is a protecting group, it is removed to obtain ezetimibe.

In step (a) above, a compound of formula (II) can be prepared by the following three or four steps:

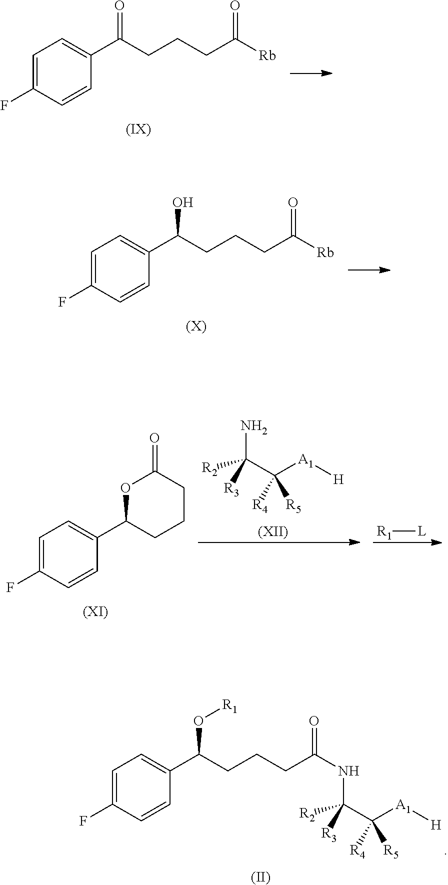

As shown in the above scheme, these steps include: (1) reducing a compound of formula (IX) to a compound of formula (X), in which Rb is OH or a leaving group, (2) cyclizing the compound of formula (X) to obtain a compound of formula (XI), (3) reacting the compound of formula (XI) with a compound of formula (XII) and, (4) if necessary, reacting the product of step (3) with R₁-L subsequently, in which L is a leaving group, to obtain a compound of formula (II).

The conversion of a compound of formula (II) to a compound of formula (III) can be achieved via one of the two routes described below:

In one route, a compound of formula (II) is first reacted with a compound having the following formula

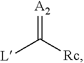

in which each of L′ and Rc is a leaving group, to obtain a compound of formula (VII) or formula (VIII):

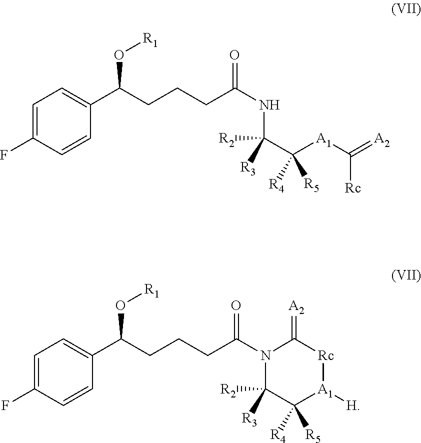

The compound of formula (VII) or formula (VIII) is subsequently cyclized to form a compound of formula (III).

In the other route, a compound of formula (II) is directly converted to a compound of formula (III) in the presence of

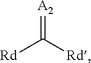

a cyclization agent, in which each of Rd and Rd′, independently, is halo, alkoxy, aryloxy, heteroaryl, or heteroaryloxy.

A leaving group, e.g., Rb, Rc, L, and L′ described above, can depart, upon direct displacement or ionization, with the pair of electrons from one of its covalent bonds (see, e.g., F. A. Carey and R. J. Sundberg, Advanced Organic Chemistry, 5th Ed., Springer, 2007). Examples include, but are not limited to, methoxy, ethoxy, tert-butoxy, tert-butyrate, methanesulfonate, triflate, p-toluenesulfonate, iodide, bromide, chloride, and trifluoroacetate.

The term “protecting group” refers to a group that, upon being attached to an active moiety (e.g., hydroxyl), prevents this moiety from interference with a subsequent reaction and can be readily removed after the reaction. Examples of a hydroxyl protecting group include, but are not limited to, alkyl, benzyl, allyl, trityl (i.e., triphenylmethyl), acyl (e.g., benzoyl, acetyl, or HOOC—Z—CO—, Z being alkylene, alkenylene, cycloalkylene, or arylene), silyl (e.g., trimethylsilyl, triethylsilyl, and t-butyldimethylsilyl), alkoxylcarbonyl, aminocarbonyl (e.g., dimethylaminocarbonyl, methylethylaminocarbonyl, and phenylaminocarbonyl), alkoxymethyl, benzyloxymethyl, and alkylmercaptomethyl. More examples are described in T. W. Greene and P. G. M. Wuts, Greene's Protective Groups in Organic Synthesis, 4th Ed., John Wiley and Sons (2007).

The term “alkyl” refers to a saturated, linear or branched hydrocarbon moiety, such as —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃) CH₃, or —C(CH₃)₃. The term “alkenyl” refers to a linear or branched hydrocarbon moiety that contains at least one double bond, such as —CH═CH—CH₃. The term “alkynyl” refers to a linear or branched hydrocarbon moiety that contains at least one triple bond, such as —C≡C—CH₃. The term “cycloalkyl” refers to a saturated, cyclic hydrocarbon moiety, such as cyclopentyl and cyclohexyl. The term “cycloalkenyl” refers to a non-aromatic, cyclic hydrocarbon moiety that contains at least one double bond, such as cyclohexenyl. The term “heterocycloalkyl” refers to a saturated, cyclic moiety having at least one ring heteroatom (e.g., N, O, or S), such as 4-tetrahydropyranyl. The term “heterocycloalkenyl” refers to a non-aromatic, cyclic moiety having at least one ring heteroatom (e.g., N, O, or S) and at least one ring double bond, such as pyranyl. The term “aryl” refers to a hydrocarbon moiety having one or more aromatic rings. Examples of aryl moieties include phenyl (Ph), naphthyl, naphthylene, pyrenyl, anthryl, and phenanthryl. The term “heteroaryl” refers to a moiety having one or more aromatic rings that contain at least one heteroatom (e.g., N, O, or S). Examples of heteroaryl moieties include furyl, furylene, fluorenyl, pyrrolyl, thienyl, oxazolyl, imidazolyl, thiazolyl, pyridyl, pyrimidinyl, quinazolinyl, quinolyl, isoquinolyl and indolyl.

Alkyl, alkenyl, alkynyl, cycloalkyl, cycloalkenyl, heterocycloalkyl, heterocycloalkenyl, aryl, and heteroaryl mentioned herein include both substituted and unsubstituted moieties, unless specified otherwise. Possible substituents on cycloalkyl, cycloalkenyl, heterocycloalkyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁° alkylamino, C₁-C₂₀ dialkylamino, arylamino, diarylamino, C₁-C₁₀ alkylsulfonamino, arylsulfonamino, C₁-C₁₀ alkylimino, arylimino, C₁-C₁₀ alkylsulfonimino, arylsulfonimino, hydroxyl, halo, thio, C₁-C₁₀ alkylthio, arylthio, C₁-C₁₀ alkylsulfonyl, arylsulfonyl, acylamino, aminoacyl, aminothioacyl, amido, amidino, guanidine, ureido, thioureido, cyano, nitro, nitroso, azido, acyl, thioacyl, acyloxy, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl can also be fused with each other.

The details of one or more embodiments of the invention are set forth in the description below. Other features, objects, and advantages of the invention will be apparent from the description and the claims.

### DETAILED DESCRIPTION

The method of preparing ezetimibe according to this invention is described in detail in this section. Below is a synthetic scheme illustrating an embodiment of this invention:

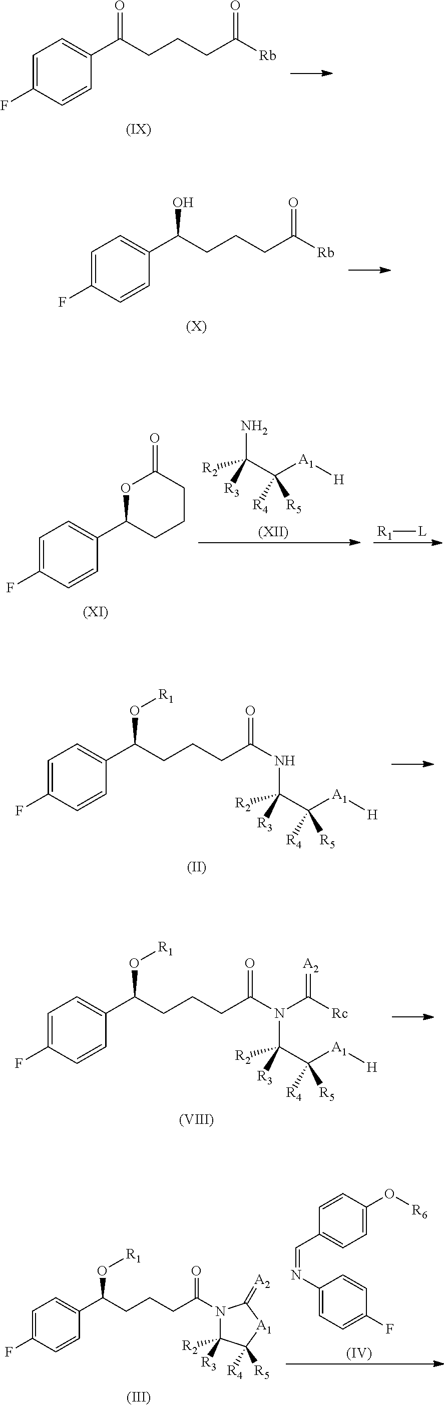

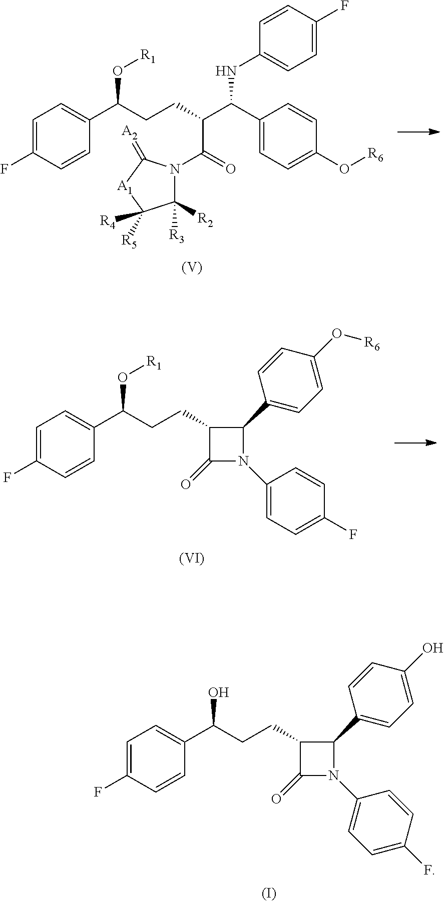

Ketone IX, e.g., 5-(4-fluorophenyl)-5-oxopentanoic acid and methyl 5-(4-fluorophenyl)-5-oxopentanoate, is stereoselectively reduced to alcohol X in the presence of a chiral catalyst, e.g., Corey-Bakshi-Shibata catalyst, or a chiral reducing agent, e.g., microbial reductase and (−)-diisopinocamphenylchloroborane (hereinafter “DIPC1”). See Bodi et al., U.S. Pat. No. 8,178,665 (2012); Homann et al., U.S. Pat. No. 5,618,707 (1997); and Kumar et al., International Application Publication WO 2005/066120 A2. Alternatively, ketone IX is reduced to a racemic mixture of alcohol X and its enantiomer, using a reducing agent, e.g., sodium borohydride. Alcohol X is then separated from the racemic mixture via a chiral resolution method, e.g., crystallization and chiral column chromatography.

Alcohol X is then cyclized to obtain lactone XI using an organic or inorganic acid, a dehydrating agent, a salt of a weak base, or a combination thereof. See Kumar et al., International Application Publication WO 2005/066120 A2. Examples of the acid include sulfuric acid, hydrochloric acid, trifluoroacetic acid, acetic acid, p-toluenesulfonic acid, and methanesulfonic acid. Examples of the dehydrating agent include molecular sieves and dicyclohexylcarbodiimide. Examples of the salt include pyridinium p-tolunenesulfonate and pyridine hydrobromide.

Subsequently, lactone XI is reacted with amine XII, e.g., (S)-(−)-2-phenylglycinol, to form amide XIII:

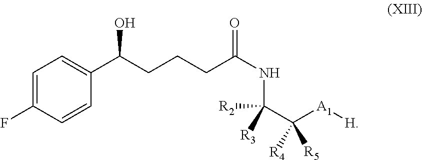

Note that Alcohol X can be reacted with amine XII to form amide XIII directly via an reaction between —C(O)Rb of alcohol X and —NH₂ of a mine XII as shown below:

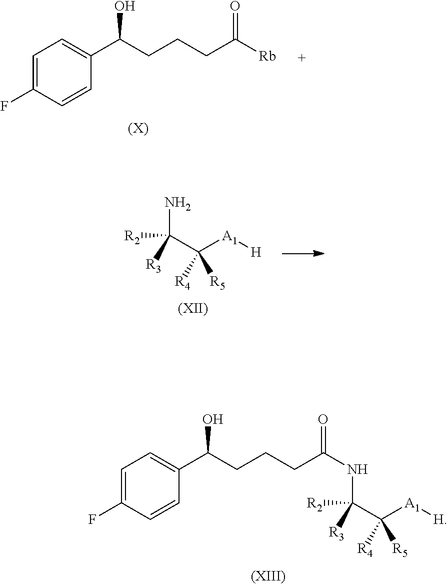

Optionally, the benzyl OH functional group in amide XIII is protected using R₁-L to obtain a compound of formula (II), in which R₁ is a hydroxyl protecting group as described in the Summary section. When amide XIII is prepared by the amidation reaction of alcohol X and amine XII, the protection step can be performed before the amidation reaction. Alternatively, the protection group can be introduced to a compound of formula (III), a compound of formula (V), or a compound of formula (VIII).

A compound of formula (II) is reacted with compound

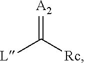

in which L″ is a leaving group as described above, to yield a compound of formula (VIII). This reaction, well known in the art, forms an amide or thioamide bond. See Iwai et al., Bioorganic and Medicinal Chemistry Letter, 21, 2812-15 (2011); and Naidu, US Patent Application Publication 2005/0192445. The compound of formula (II) can also be reacted with compound

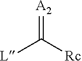

to form a compound of formula (VII). Both of the compound of formula (VII) and the compound of formula (VIII) can be subsequently cyclized to obtain a compound of formula (III) using a catalyst, e.g., NaH, tert-butoxide, and SinO₂, that facilitates the formation of amides or esters. See Ito et al., Tetrahedron Letters, 44, 7949-52 (2003); Lee, et al., Bioorganic & Medicinal Chemistry, 15, 3499-3504 (2007); Fukatsu et al., EP 1,661,898 (2006); and Feldman et al., Journal of Organic Chemistry, 67, 7096-7109 (2002).

As described above, a compound of formula (II) can be converted to a compound of formula (III) in one step in the presence of a cyclization agent. Examples of the cyclization agent include:

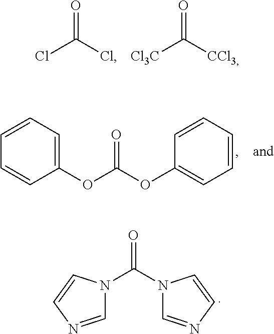

A compound of formula (III) is then reacted with a compound of formula (IV) to yield a compound of formula (V), which is subsequently converted to a compound of formula (VI) via a cyclization reaction. When each of R₁ and R₆ in a compound of formula (VI) is not H, they are de-protected to obtain ezetimibe. For preparing ezetimibe from a compound of formula (III), see Thiruvengadam et al., U.S. Pat. No. 5,561,227 (1996); and Bodi et al., U.S. Pat. No. 8,178,665 (2012).

The method of this invention has several advantages. It includes a step of reducing a carbonyl group to a hydroxyl group before the formation of an oxazolidinone intermediate, thus improving the efficiency. Further, inexpensive reducing agents can be used in this step. In addition, the method includes a novel intermediate, i.e., a compound of formula (II), which can be easily purified by crystallization.

Ezetimibe was prepared following the exemplary procedure described below. This specific example is to be construed as merely illustrative, and not limitative of the remainder of the disclosure in any way whatsoever. Without further elaboration, it is believed that one skilled in the art can, based on the description herein, utilize the present invention to its fullest extent. All publications cited herein are incorporated by reference in their entirety.

### Preparation of 5-(4-fluorophenyl)-5-oxopentanoic acid

As shown below, 5-(4-fluorophenyl)-5-oxopentanoic acid (i.e., compound 3) was prepared from fluorobenzene (i.e., compound 1) and glutaric anhydride (i.e., compound 2):

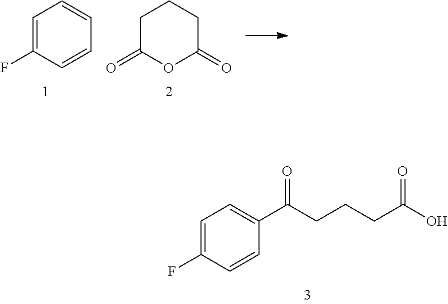

To a suspension of aluminum chloride (205.85 g, 1.54 mol) in dichloromethane (500 mL) was added a solution of glutaric anhydride (80 g, 0.7 mol) in dichloromethane (125 mL) at 0° C. The reaction mixture was stirred for 30 minutes. Fluorobenzene (67.36 g, 0.7 mol) was then added slowly. The progress of the reaction was monitored by TLC. Upon completion, the reaction mixture was poured into ice water (2000 mL) to precipitate a crude solid product, which was collected by filtration. The crude was re-dissolved in a 3% aqueous sodium hydroxide solution (1100 mL). After being washed with dichloromethane (300 mL), the aqueous solution was acidified to obtain a solid product. The product was filtered, washed with water, and vacuum dried to yield compound 3 (125 g).

HNMR of compound 3 (CDCl₃, 300M Hz): δ=2.10 (q, J=7.2 Hz, 2H), 2.51 (t, J=7.2 Hz, 2H), 3.65 (t, J=7.2 Hz, 2H), 7.13 (t, J=7.4 Hz, 2H), 7.98 (q, J=5.4 Hz, 2H).

### Preparation of methyl 5-(4-fluorophenyl)-5-oxopentanoate

As shown below, compound 3 was converted to methyl 5-(4-fluorophenyl)-5-oxopentanoate, i.e., compound 4:

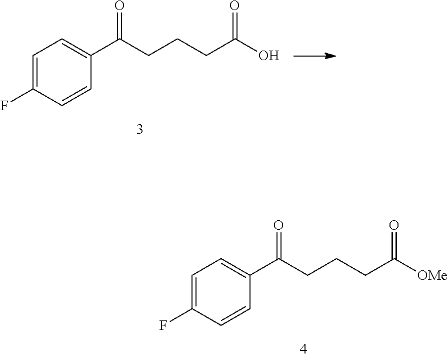 [SMILES: O=C(O)CCCC(=O)c1ccc(F)cc1 ; COC(=O)CCCC(=O)c1ccc(F)cc1]

To a flask were added 3 g of compound 3, 30 mL of methanol, and 0.25 mL of concentrated H₂SO₄. After the resultant solution was heated to reflux for 1 hour, it was cooled and then concentrated to ⅕ of its volume. Ethyl acetate (100 mL) was added. The mixture was washed with a saturated NaHCO₃ aqueous solution and a brine solution. The ethyl acetate layer was separated and concentrated to dryness to give compound 4 in 100% yield.

HNMR of compound 4 (CDCl₃, 300M Hz): δ=2.01 (q, J=7.2 Hz, 2H), 2.45 (t, J=7.2 Hz, 2H), 3.03 (t, J=7.2 Hz, 2H), 3.68 (s, 3H), 7.13 (t, J=8.7 Hz, 2H), 7.99 (q, J=5.4 Hz, 2H).

### Preparation of (S)-5-(4-fluorophenyl)-5-hydroxypentanoic acid

As shown below, compound 4 was reduced to (S)-5-(4-fluorophenyl)-5-hydroxypentanoic acid, i.e., compound 5:

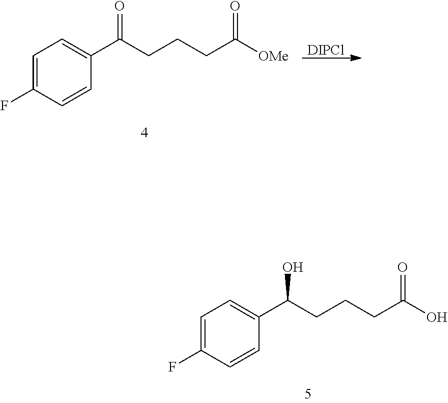

To a flask were added NaBH₄ (4.6 g), dimethoxyethane (72.5 mL), and α-pinene (77 mL). After the resultant milky mixture was cooled to −15° C., BCl₃ (1 mol in 133 mL of hexanes) was added dropwise over 20 minutes. The mixture was stirred for another 20 minutes at −15° C., still another 20 minutes at 0° C., 1 hour at 20-25° C., and another 1 hour at 40° C. It was then cooled to −15° C. Compound 4 (31 g in 138 mL of tetrahydrofuran) was added dropwise at −10 to −15° C. The mixture was then stirred at 4° C. for 16 hours. Water (133 mL) was added dropwise at 4° C. followed by a 5 N NaOH aqueous solution (380 mL). The resultant basic solution was slowly warmed to ambient temperature and then stirred for 2 hours. An aqueous NaHCO₃ solution (250 mL) and dichloromethane (412 mL) were added and stirred for 15 minutes. The aqueous layer was separated and acidified to precipitate a solid product. The product was filtered and dried to give 25 g of compound 5.

HNMR of compound 5 (CDCl₃, 300M Hz): δ=1.82-1.90 (m, 1H), 1.92-2.03 (m, 2H), 2.10-2.17 (m, 1H), 2.50-2.76 (m, 2H), 5.30 (dd, J=3 Hz, 1H), 7.05 (t, J=7.4 Hz, 2H), 7.32 (q, J=5.4 Hz, 2H).

### Preparation of (S)-6-(4-fluorophenyl)tetrahydro-2H-pyran-2-one

As shown below, compound 5 was cyclized to obtain (S)-6-(4-fluorophenyl)tetrahydro-2H-pyran-2-one, i.e., compound 6:

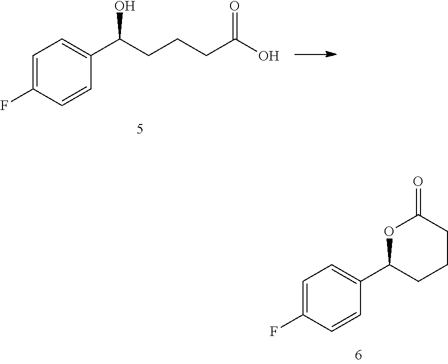 [SMILES: O=C(O)CCC[C@H](O)c1ccc(F)cc1 ; O=C1CCC[C@@H](c2ccc(F)cc2)O1]

To a flask were added compound 5 (24.9 g), dichloromethane (125 mL), and trifluoroacetic acid (1.3 mL). The resultant organic solution was stirred at ambient temperature for 3 hours. It was then washed with a saturated NaHCO₃ solution twice (50 ml x 2) and a brine solution once (50 mL), dried with a drying agent, concentrated, and filtered to collect 21.5 g of compound 6 as a light yellowish solid.

HNMR of compound 6 (CDCl₃, 300M Hz): δ=1.86-1.91 (m, 1H), 1.95-2.04 (m, 2H), 2.12-2.19 (m, 1H), 2.52-2.58 (m, 1H), 2.60-2.77 (m, 1H), 5.32 (dd, J=3.6 Hz, 1H), 7.06 (q, J=6.0 Hz, 2H), 7.32 (q, J=3.3 Hz, 2H).

### Preparation of (S)-5-(4-fluorophenyl)-5-hydroxy-N—((S)-2-hydroxy-1-phenylethyl)pentanamide

As shown below, the lactone ring of compound 6 was opened by (S)-(+)-phenylglycinol to prepare (S)-5-(4-fluorophenyl)-5-hydroxy-N—((S)-2-hydroxy-1-phenylethyl)pentanamide, i.e., compound 7:

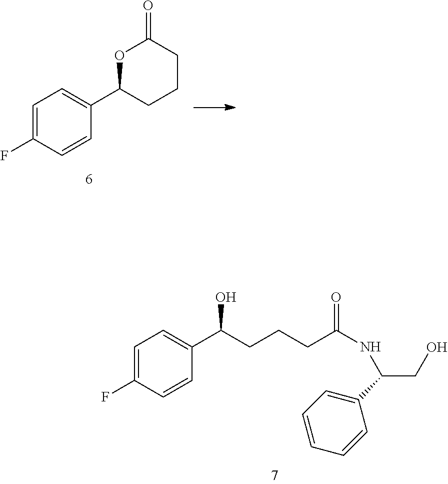 [SMILES: O=C1CCC[C@@H](c2ccc(F)cc2)O1 ; O=C(CCC[C@H](O)c1ccc(F)cc1)N[C@H](CO)c1ccccc1]

To a flask were sequentially added compound 6 (0.97 g), (S)-(+)-phenylglycinol (0.72 g), 4-dimethylaminopyridine (0.31 g), and dioxane (5 mL). The resultant solution was heated to 55-60° C. and stirred for 16 hours. After it was cooled to ambient temperature, dichloromethane (15 mL) was added. The solution was then washed with an aqueous NaH₂PO₄ solution (5 mL), a saturated NaHCO₃ solution (5 mL), and a brine solution (5 mL). It was dried, concentrated, and filtered to collect 1.5 g of compound 7. Unexpectedly, compound 7 was obtained as crystal with a high chiral purity.

HNMR of compound 7 (CDCl₃, 300M Hz): δ=1.74-1.82 (m, 4H), 2.32 (t, J=6.6 Hz, 2H), 2.84 (br, 1H), 2.98 (br, 1H), 3.82 (s, 2H), 4.67 (br, 1H), 5.076 (dd, J=5.1 Hz, 1H), 6.394 (d, J=7.2 Hz, 1H), 7.018 (t, J=8.7 Hz, 2H), 7.253-7.387 (m, 7H).

### Preparation of tert-butyl (S)-5-(4-fluorophenyl)-5-hydroxypentanoyl((S)-2-hydroxy-1-phenylethyl)carbamate

As shown below, compound 7 was converted to tert-butyl (S)-5-(4-fluorophenyl)-5-hydroxypentanoyl((S)-2-hydroxy-1-phenylethyl)carbamate, i.e., compound 8:

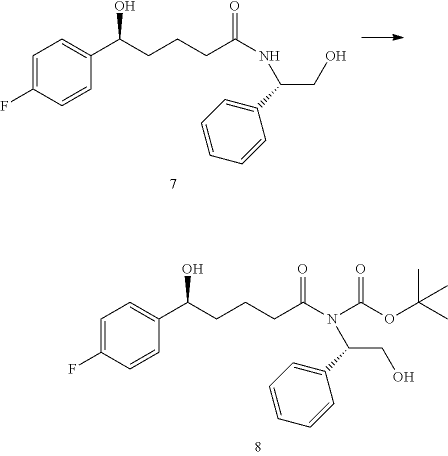 [SMILES: O=C(CCC[C@H](O)c1ccc(F)cc1)N[C@H](CO)c1ccccc1 ; CC(C)(C)OC(=O)N(C(=O)CCC[C@H](O)c1ccc(F)cc1)[C@H](CO)c1ccccc1]

To a flask were added compound 7 (1.0 g), 4-dimethylaminopyridine (73 mg), di-tert-butyl carbonate (0.98 g), and tetrahydrofuran (6 mL). The resultant solution was cooled to 4° C. Triethylamine (0.85 mL) was added. After 1 hour, additional di-tert-butyl carbonate (0.2 g) was added. The solution was stirred for another 1 hour, diluted with ethyl acetate (30 ml), and washed with an aqueous NaH₂PO₄ solution twice (20 mL x 2), an aqueous NaHCO₃ solution once (10 mL), and a brine solution once (10 mL). It was then concentrated, dried, and filtered to collect 1.27 g of compound 8.

HNMR of compound 8 (CDCl₃, 300M Hz): δ=1.44 (s, 9H), 1.67-1.75 (m, 4H), 2.27 (q, J=3.9 Hz, 2H), 2.40 (d, J=3.3 Hz, 1H), 4.24 (dd, J=4.2 Hz, 2H), 4.64 (t, 1H), 5.28 (q, J=7.2 Hz, 1H), 6.28 (d, J=8.1 Hz, 1H), 7.00 (t, J=8.7 Hz, 2H), 7.24-7.32 (m, 7H).

### Preparation of (S)-3-((S)-5-(4-fluorophenyl)-5-hydroxypentanoyl)-4-phenyloxazolidin-2-one

As shown below, compound 8 was cyclized to obtain (S)-3-((S)-5-(4-fluorophenyl)-5-hydroxypentanoyl)-4-phenyloxazolidin-2-one, i.e., compound 9:

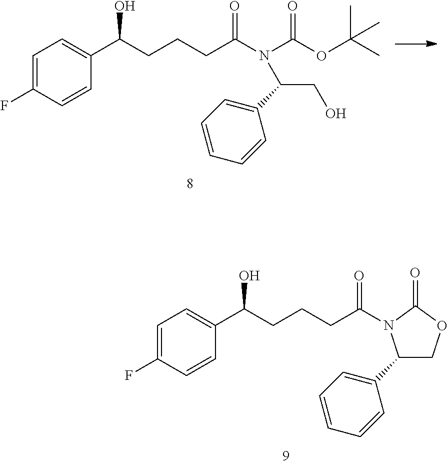 [SMILES: CC(C)(C)OC(=O)N(C(=O)CCC[C@H](O)c1ccc(F)cc1)[C@H](CO)c1ccccc1 ; O=C(CCC[C@H](O)c1ccc(F)cc1)N1C(=O)OC[C@@H]1c1ccccc1]

Compound 8 (1.29 g) was dissolved in dimethylformamide (2 mL). After the solution was cooled to 4° C., NaH (13 mg) was added. The resultant reaction mixture was warmed to ambient temperature, and then stirred for 2 hours. After the reaction was quenched with an aqueous saturated solution of ammonium chloride (50 mL), ethyl acetate was used to extract the reaction mixture twice (100 mL x 2). The ethyl acetate extracting solutions were combined, dried with anhydrous Na₂SO₄, and filtered. The filtrate was concentrated to give a crude product (2.0 g), which was purified by column chromatography to yield 800 mg of compound 9.

HNMR of compound 9 (CDCl₃, 300M Hz): δ=1.62-1.74 (m, 4H), 1.98 (d, J=3.6 Hz, 1H), 2.97 (t, J=6.6 Hz, 2H), 4.26 (q, J=3.6 Hz, 1H), 4.65 (m, 2H), 5.39 (dd, J=3.6 Hz, 1H), 7.0 (t, J=8.7 Hz, 2H), 7.24-7.41 (m, 7H).

### Preparation of (S)-3-((2R,5S)-5-(4-fluorophenyl)-2-((R)-(4-fluorophenylamino)(4-(trimethylsilyloxy)phenyl)methyl)-5-(trimethylsilyloxy)pentanoyl)-4-phenyloxazolidin-2-one

As shown below, compound 9 was reacted with 4-((4-fluorophenylimino)methyl)phenol, i.e., compound 10, to obtain (S)-3-((2R,5S)-5-(4-fluorophenyl)-2-((R)-(4-fluorophenylamino)(4-(trimethylsilyloxy)phenyl)methyl)-5-(trimethylsilyloxy)pentanoyl)-4-phenyloxazolidin-2-one, i.e., compound 11:

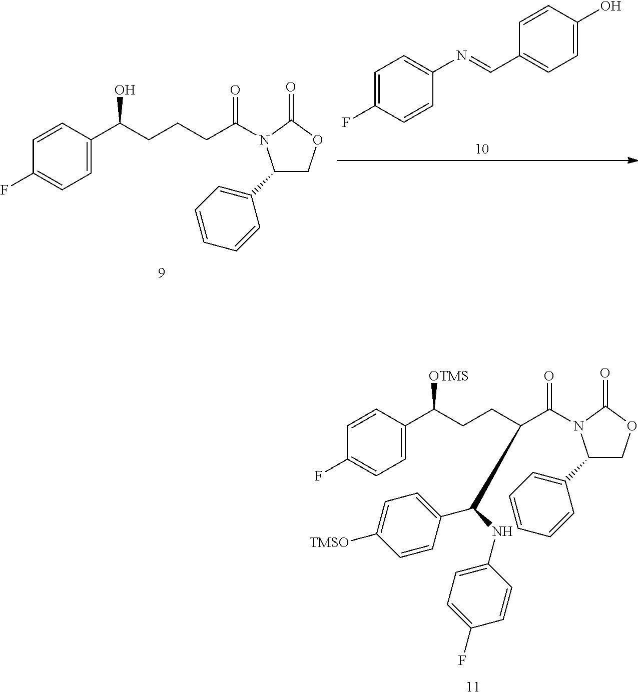

Compounds 9 (0.8 g) and 10 (1.0 g) were dissolved in dichloromethane (15 mL). After the dichloromethane solution was cooled to −10° C., N,N-diisopropylethylamine (2.6 mL) was added, followed by dropwise addition of trimethylsilyl chloride (1.3 mL) over a period of 10 minutes. The mixture was stirred for 1 hour and cooled to −30° C. TiCl₄ (0.31 ml) was then added dropwise at −30 to −25° C. The reaction was complete after 2 hours as indicated by TLC. Acetic acid (0.8 mL) was slowly added with the temperature kept below −25° C., and the mixture thus formed was poured into a 7% aqueous solution of tartaric acid (12 mL) at 0° C. After it was warmed to ambient temperature, two separated layers, an organic layer and an aqueous layer, formed. The organic layer was separated, washed with water, and concentrated to dryness to yield a residue, which was dissolved in dichloromethane (10 mL), together with bis(trimethylsily)acetamide (1 mL). The resultant solution was heated to reflux for 2 hours. After the reaction was complete, the solvent was evaporated to afford compound 11, which was used in the next step without purification.

HNMR of compound 11 (300 MHz, CDCl₃): δ 7.44-7.28 (m, 5H), 7.22-7.01 (m, 9H), 6.96 (t, J=8.8 Hz, 2H), 6.83 (d, J=8.6 Hz, 2H), 6.71 (t, J=8.6 Hz, 2H), 6.35 (M, 2H), 5.39 (dd, J=8.4 Hz, J=3.3 Hz, 1H), 5.00 (s, 2H), 4.84 (d, J=9.8 Hz, 1H), 4.61 (t, J=8.7 Hz, 1H), 4.58-4.45 (m, 2H), 4.30 (t, J=9.1 Hz, 1H), 4.16 (dd, J=8.8 Hz₅ J=3.3 Hz, 1H), 1.86 (d, 0.1=3.5 Hz, 1H), 1.81-1.54 (m, 3H), 1.43 (m, 1H).

### Preparation of Ezetimibe

As shown below, compound 11 was cyclized to obtain ezetimibe:

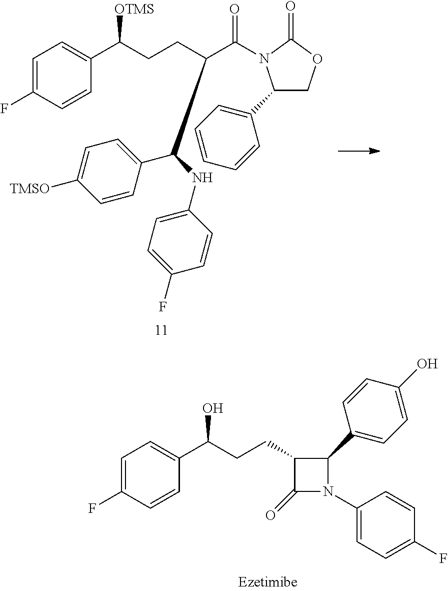

Compound 11 obtained in the above step, along with tetrabutylammonium fluoride trihydrate (5 mg) and bis(trimethylsilyl)acetamide (1 mL), was dissolved in tert-butyl methyl ether (25 mL). The mixture was stirred at ambient temperature for 1.5 hours and then concentrated to dryness to give a residue, which was dissolved in ethyl acetate (20 mL) and 1 N H₂SO₄ (2 mL). The resultant mixture in ethyl acetate was stirred at ambient temperature for 30 minutes and then allowed to sit until it separated into an organic layer and an aqueous layer. The organic layer was separated, washed with brine, dried with anhydrous Na₂SO₄, and filtered. The filtrate was concentrated to give a residue, which was purified by column chromatography to obtain 0.6 g of ezetimibe.

HNMR (300 MHz, DMSO-d₆): δ 1.73-1.88 (m, 4H), 3.08 (m, 1H), 4.50 (d, 1H, 3.4), 4.79 (d, 1H, 2.1), 5.29 (d, 1H, 4.1), 6.77 (d, 2H, 8.6), 7.07-7.33 (m, 10H) 9.54 (s, 1H).

¹³CNMR (75 MHz, DMSO-d₆): 824.6, 36.4, 59.5, 59.6, 71.1, 114.7 (21 Hz), 115.8, 115.9 (23 Hz), 118.3 (8 Hz), 127.6 (8 Hz), 127.6, 127.9, 130.0 (2 Hz), 142.2 (2 Hz), 157.9 (227 Hz), 157.5, 161.3 (228 Hz), 167.4.

C₂₄H₂₁F₂NO₃ M.W. 409.43

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method of preparing a compound of formula (I): 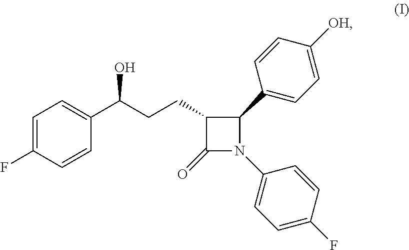 [SMILES: O=C1[C@H](CC[C@H](O)c2ccc(F)cc2)[C@@H](c2ccc(O)cc2)N1c1ccc(F)cc1]
the method comprising:
(a) converting a compound of formula (II) to a compound of formula (III) as shown below: 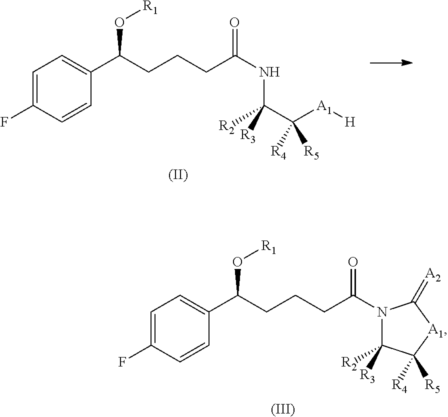
in which
R₁ is H or a protecting group;
R₂ is C₁-C₁₀ alkyl, C₂-C₁₀ alkenyl, C₂-C₁₀ alkynyl, C₃-C₁₀ cycloalkyl, C₃-C₁₀ cycloalkenyl, C₁-C₁₀ heterocycloalkyl, C₁-C₁₀ heterocycloalkenyl, aryl, or heteroaryl;
each of R₃, R₄, and R₅, independently, is H, C₁-C₁₀ alkyl, C₂-C₁₀ alkenyl, C₂-C₁₀ alkynyl, C₃-C₁₀ cycloalkyl, C₃-C₁₀ cycloalkenyl, C₁-C₁₀ heterocycloalkyl, C₁-C₁₀ heterocycloalkenyl, aryl, or heteroaryl, R₂, R₃, R₄, and R₅, together, determining the stereochemistry of step (b) below;
A₁ is O, S, or NRₐ, Rₐ being C₁-C₁₀ alkyl, C₂-C₁₀ alkenyl, C₂-C₁₀ alkynyl, C₃-C₁₀ cycloalkyl, C₃-C₁₀ cycloalkenyl, C₁-C₁₀ heterocycloalkyl, C₁-C₁₀ heterocycloalkenyl, aryl, or heteroaryl; and
A₂ is O or S;
(b) reacting the compound of formula (III) with a compound of formula (IV) to obtain a compound of formula (V) as shown below: 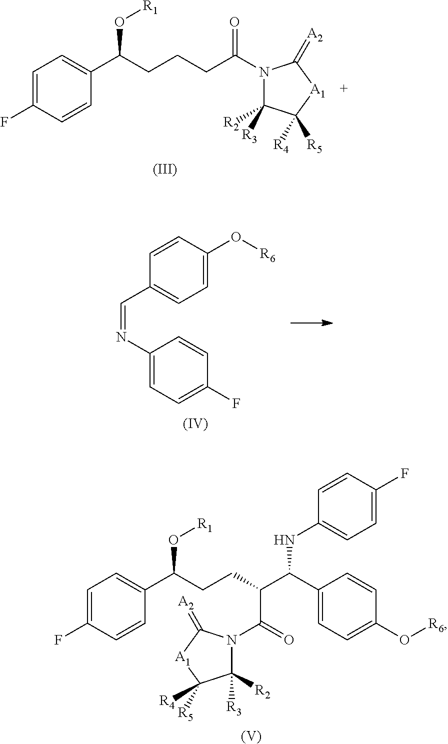
in which R₆ is H or a protecting group;
(c) cyclizing the compound of formula (V) to obtain a compound of formula (VI): 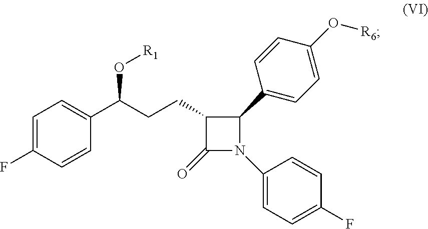
and
(d) if either R₁ or R₆ is a protecting group, removing the protecting group to obtain the compound of formula (I).

2. The method of claim 1, wherein A₁ is O or S.

3. The method of claim 2, wherein R₂ is methyl, ethyl, propyl, isopropyl, butyl, isobutyl, tert-butyl, phenyl, benzyl, or diphenylmethyl; and each of R₃, R₄, and R₅ is H.

4. The method of claim 3, wherein R₂ is isopropyl or phenyl.

5. The method of claim 4, wherein the compound of formula (II) is prepared by the following steps shown below: 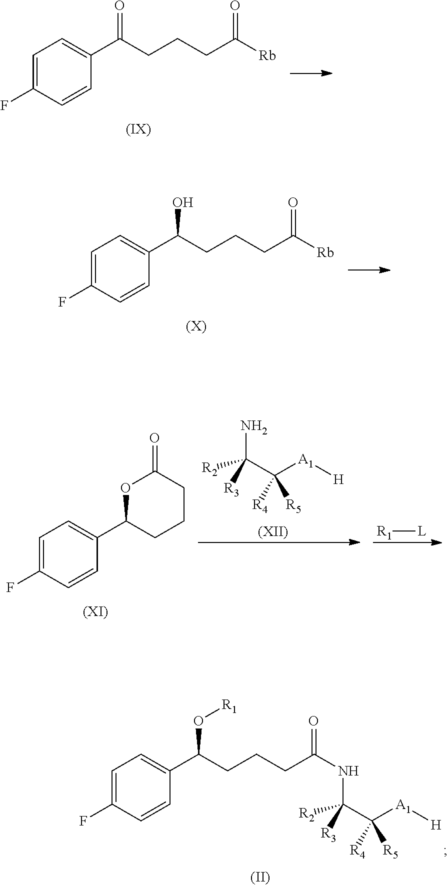
the steps comprising:
(1) reducing a compound of formula (IX) to a compound of formula (X),
(2) cyclizing the compound of formula (X) to obtain a compound of formula (XI),
(3) reacting the compound of formula (XI) with a compound of formula (XII), and
(4) if necessary, reacting the product of step (3) with R₁-L to obtain the compound of formula (II),
in which L is a leaving group and Rb is OH or a leaving group.

6. The method of claim 2, wherein each of A₁ and A₂ is O.

7. The method of claim 6, wherein R₂ is methyl, ethyl, propyl, isopropyl, butyl, isobutyl, tert-butyl, phenyl, benzyl, or diphenylmethyl; and each of R₃, R₄, and R₅ is H.

8. The method of claim 7, wherein R₂ is isopropyl or phenyl.

9. The method of claim 8, wherein the compound of formula (II) is prepared by the following steps shown below: 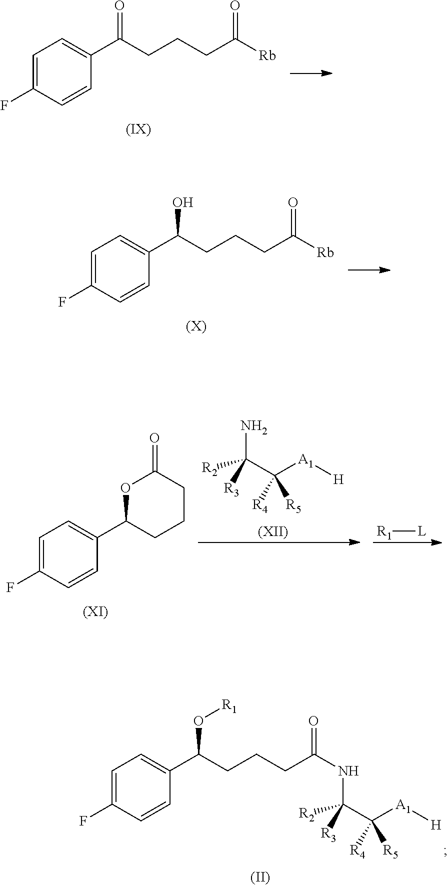
the steps comprising:
(1) reducing a compound of formula (IX) to a compound of formula (X),
(2) cyclizing the compound of formula (X) to obtain a compound of formula (XI),
(3) reacting the compound of formula (XI) with a compound of formula (XII), and
(4) if necessary, reacting the product of step (3) with R₁-L to obtain the compound of formula (II),
in which L is a leaving group and Rb is OH or a leaving group.

10. The method of claim 1, wherein R₂ is methyl, ethyl, propyl, isopropyl, butyl, isobutyl, tert-butyl, phenyl, benzyl, or diphenylmethyl; and each of R₃, R₄, R₅, and R₆ is H.

11. The method of claim 8, wherein R₂ is isopropyl or phenyl.

12. The method of claim 1, wherein the compound of formula (II) first reacts with a compound having the following formula: 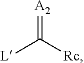
in which each of L′ and Rc is a leaving group, to obtain a compound of formula (VII) or (VIII): 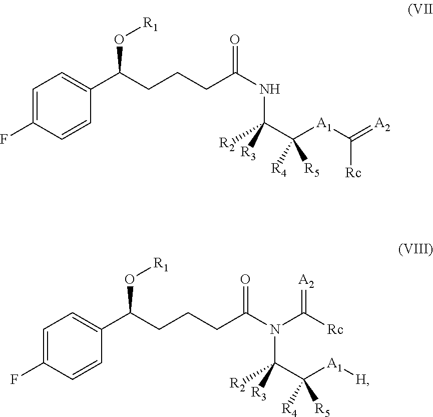
and the compound of formula (VII) or (VIII) is subsequently cyclized to form the compound of formula (III).

13. The method of claim 12, wherein A₁ is O or S; R₂ is methyl, ethyl, propyl, isopropyl, butyl, isobutyl, tert-butyl, phenyl, benzyl, or diphenylmethyl; and each of R₃, R₄, R₅ and R₆ is H.

14. The method of claim 13, wherein each of A₁ and A₂ is O, R₂ is isopropyl or phenyl.

15. The method of claim 14, wherein the compound of formula (II) is prepared by the following steps shown below: 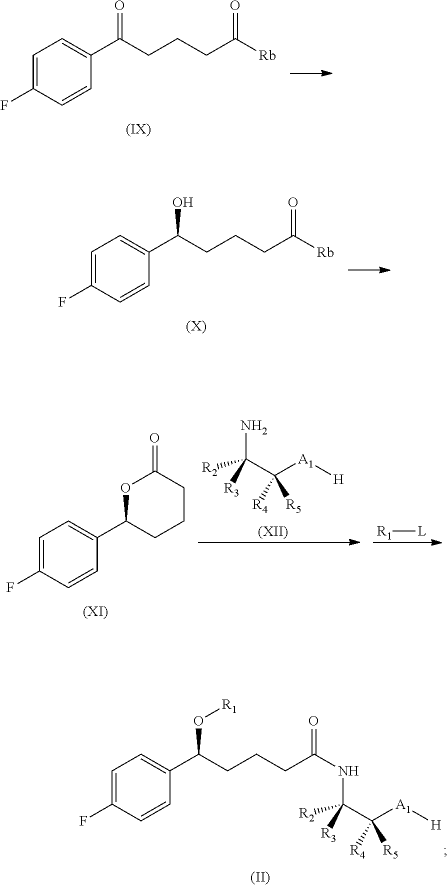
the steps comprising:
(1) reducing a compound of formula (IX) to a compound of formula (X),
(2) cyclizing the compound of formula (X) to obtain a compound of formula (XI),
(3) reacting the compound of formula (XI) with a compound of formula (XII), and
(4) if necessary, reacting the product of step (3) with R₁-L to obtain the compound of formula (II),
in which L is a leaving group and Rb is OH or a leaving group.

16. The method of claim 1, wherein the compound of formula (II) is directly converted to the compound of formula (III) in the presence of 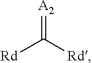
in which each of Rd and Rd′, independently, is halo, alkoxy, aryloxy, heteroaryl, or heteroaryloxy.

17. The method of claim 16, wherein A₁ is O or S; R₂ is methyl, ethyl, propyl, isopropyl, butyl, isobutyl, tert-butyl, phenyl, benzyl, or diphenylmethyl; and each of R₃, R₄, R₅ and R₆ is H.

18. The method of claim 17, wherein each of A₁ and A₂ is O and R₂ is isopropyl or phenyl.

19. The method of claim 18, wherein the compound of formula (II) is prepared by the following steps shown below: 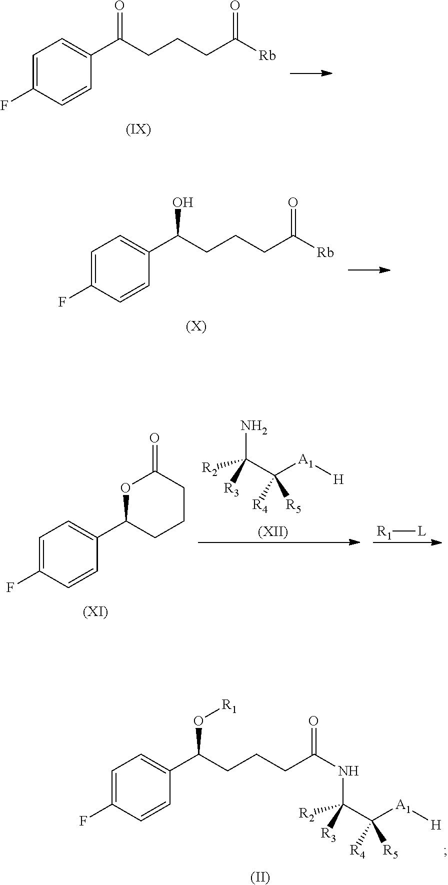
the steps comprising:
(1) reducing a compound of formula (IX) to a compound of formula (X),
(2) cyclizing the compound of formula (X) to obtain a compound of formula (XI),
(3) reacting the compound of formula (XI) with a compound of formula (XII), and
(4) if necessary, reacting the product of step (3) with R₁-L to obtain the compound of formula (II),
in which L is a leaving group and Rb is OH or a leaving group.

20. The method of claim 1, wherein the compound of formula (II) is prepared by the following steps shown below: 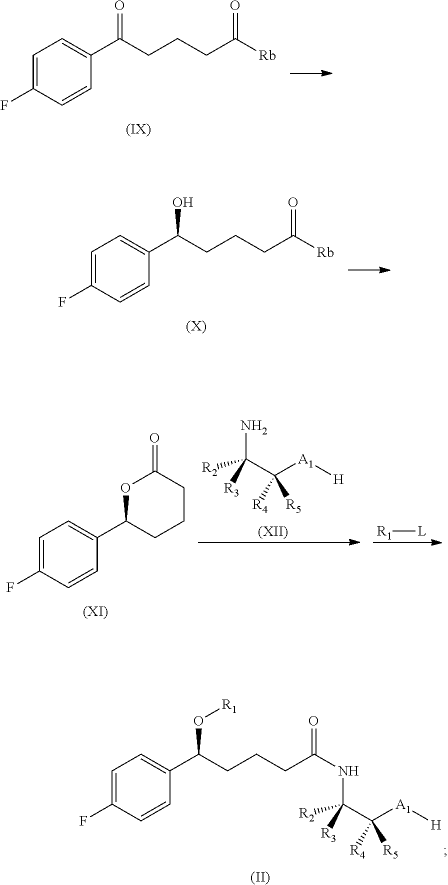
the steps comprising:
(1) reducing a compound of formula (IX) to a compound of formula (X),
(2) cyclizing the compound of formula (X) to obtain a compound of formula (XI),
(3) reacting the compound of formula (XI) with a compound of formula (XII), and
(4) if necessary, reacting the product of step (3) with R₁-L to obtain the compound of formula (II),
in which L is a leaving group and Rb is OH or a leaving group.
